(19) United States
(12) Patent Application Publication
(10) Pub. No.: US 2017/0050943 A1
(43) Pub. Date: Feb. 23, 2017

(54) METHOD FOR PRODUCING FURFURAL, AND METHOD FOR PRODUCING FURAN

(71) Applicant: MITSUBISHI CHEMICAL CORPORATION, Chiyoda-ku (JP)

(72) Inventors: Yusuke IZAWA, Mie (JP); Norikazu Konishi, Mie (JP); Yosuke Suzuki, Mie (JP)

(73) Assignee: MITSUBISHI CHEMICAL CORPORATION, Chiyoda-ku (JP)

(21) Appl. No.: 15/345,838

(22) Filed: Nov. 8, 2016

Related U.S. Application Data
(63) Continuation of application No. PCT/JP2015/063228, filed on May 7, 2015.

(30) Foreign Application Priority Data
May 8, 2014 (JP) 2014-096968

(51) Int. Cl. C07D 307/36; C07D 307/48

(52) U.S. Cl. CPC C07D 307/36; C07D 307/48

(57) ABSTRACT

A problem is to provide an industrially advantageous method in which in purifying a furfural composition, the formation of a solid matter which have been unable to be controlled so far is stably reduced, and furfural is purified with high efficiency wherein the problem has been solved by a method for producing furfural including distilling a composition containing furfural by a distillation column to obtain furfural, wherein a concentration of a furfural dimer in a column bottom liquid of the distillation column is controlled to 20 ppm by mass to 5,000 ppm by mass.

### TECHNICAL FIELD

The present invention relates to a method for producing furfural and a method for producing furan and also to a method for producing furan using the thus obtained furfural.

### BACKGROUND ART

With respect to chemicals (for example, ethanol, succinic acid, 1,4-butanediol, etc.) which have hitherto been produced from petroleum, in recent years, it is investigated to produce such petrochemical derivatives using a biomass resource as a raw material without using petroleum as the raw material.

In the case where the biomass resource is a raw material, typically, the biomass resource is roughly classified into two kinds of an edible biomass resource, such as a sugar, etc., and a nonedible biomass, such as hemicellulose, cellulose, etc., with respect to the kind thereof. Furfurals formed from hemicellulose or the like become a component which inhibits the fermentation in fermentation of a biomass resource, and therefore, they have been removed as impurities so far. However, from the viewpoint of effective use of a biomass resource, a technique of producing the above-described chemicals even from furfurals which have been removed as an impurity so far is demanded.

A technique of extracting furfural from a biomass resource has been known from old. In addition, a majority of furfural is converted into furfuryl alcohol and used as a raw material of furan resins.

Besides, as a technique of producing a chemical product using furfural, for example, there is known a method in which furfural is converted into furan by means of a decarbonylation reaction, and the furan is then hydrogenated to produce tetrahydrofuran (Patent Literature 1) In addition, it is also known that there is such a problem that in furfural, oxidation is advanced in air (in a state of coming into contact with oxygen) or polymerization of furfural is advanced to generate a polymer, or others. As a method of inhibiting oxidation and polymerization of furfural, Patent Literature 2 discloses a method of introducing, as an inhibitor, an amine having an aryl group, such as a dialkylphenylenediamine, etc. In addition, with respect to a purification technique of furfural, Patent Literature 3 discloses a method in which the polymerization of furfural is inhibited to inhibit the formation of a solid matter, and high-purity furfural is efficiently distilled in a stable manner from the raw material furfural.

### CITATION LIST

### Patent Literature

Patent Literature 1: JP-A-2009-149634

Patent Literature 2: JP-A-6-329651

Patent Literature 3: JP-A-2014-12663

### SUMMARY OF INVENTION

### Technical Problem

In the case where it is contemplated to produce high-purity furfural at the industrial level, in view of the fact that furfural per se is a substance that is liable to be polymerized as described above, there was such a problem that the purity does not increase because of the formation of a solid matter in distillation. By adopting the purification means of furfural as described in the above-described Patent Literature 2 or Patent Literature 3, it has become possible to efficiently obtain high-purity furfural by inhibiting the formation of a solid matter to some extent.

However, when it is contemplated to obtain furfural having a higher purity from the viewpoint of purity or quality of furfural as a raw material in producing furan or furfuryl alcohol, there was a case where a solid matter is formed in distillation by a distillation column according to the method described in Patent Literature 2 or Patent Literature 3. Above all, when it is contemplated to obtain furfural having a higher purity and also having a small amount of impurities from a furfural-containing composition in which a compound having a higher boiling point than furfural is contained, there was a case where by-production of a solid matter is remarkably generated to impair the purification.

In view of the foregoing problems, the present invention has been made, and an object thereof is to provide a method for producing furfural including purifying a composition containing furfural, in which the formation of a solid matter which have been unable to be controlled by the conventional purification techniques is stably reduced; and purification is industrially efficiently performed, thereby producing furfural having a high purity.

### Solution to Problem

In order to solve the foregoing problem, the present inventors made extensive and intensive investigations. As a result, it has been found that a causing substance of forming a solid matter, which cannot be thoroughly removed by a conventional purification technique (for example, contact with an anion exchange resin or contact with an amine), is contained in a composition containing furfural.

Above all, in particular, it has been found that a concentration of a furfural dimer (5-(2-furanylcarbonyl)-2-furancarboxyaldehyde and/or di-2-furylethanedione) strongly correlates with the formation of a solid matter. Then, it has been found that by performing distillation with controlling the concentration of this furfural dimer to a fixed concentration range, furfural having a high purity can be stably produced with inhibiting the formation of a solid matter within a distillation column

In addition, it has also been found that by preferably controlling a concentration of furancarboxylic acid along with the furfural dimer to a fixed concentration range, it is possible to inhibit the amount of a formed solid matter.

Specifically, the gist of the present invention resides in the following [1] to [7].

[1] A method for producing furfural comprising distilling a composition comprising furfural by a distillation column to obtain furfural, wherein a concentration of a furfural dimer in a column bottom liquid of the distillation column is 20 ppm by mass to 5,000 ppm by mass.
[2] The method for producing furfural according to the above [1], wherein a concentration of furancarboxylic acid in the column bottom liquid of the distillation column is 50 ppm by mass to 8,000 ppm by mass.
[3] The method for producing furfural according to the above [1] or [2], including a step of prior to distilling the composition comprising furfural by the distillation column, concentrating a compound having a higher boiling point than furfural in crude furfural obtained after bringing the crude furfural into contact with an anion exchange resin and/or a basic compound in advance, to obtain the composition comprising furfural.
[4] The method for producing furfural according to any one of the above [1] to [3], wherein a concentration of furfural in the composition comprising furfural is 87.0% by mass or more and 99.0% by mass or less.
[5] The method for producing furfural according to any one of the above [1] to [4], wherein a temperature of the column bottom liquid of the distillation column for distilling the composition comprising furfural is 60 to 180° C.
[6] The method for producing furfural according to any one of the above [1] to [5], wherein an acid value of the column bottom liquid of the distillation column for distilling the composition comprising furfural is 10 mg-KOH/g or less.
[7] A method for producing furan comprising feeding the furfural obtained by the method for producing furfural according to any one of the above [1] to [6] into a reactor; performing a decarbonylation reaction in the presence of a catalyst to form furan; and extracting a mixed gas containing the furan as a main component from an outlet of the reactor.

In accordance with the present invention, in continuously purifying a composition containing furfural on an industrial scale to produce furfural having a high purity, a stable reduction of a solid matter can be expected. In addition, by preferably controlling a concentration of furancarboxylic acid along with a furfural dimer to a fixed concentration range, it also becomes possible to reduce a solid matter. In the case where the furfural dimer or furancarboxylic acid is present in a fixed concentration, when a distillation column is operated with controlling this concentration, furfural having a high purity can be efficiently produced with avoiding the formation of a high-boiling component and a solid matter,

In addition, in a bottom of the distillation column, heat transfer obstruction to be caused due to a stain within a forced circulation pump and a reboiler tube used as a heating source can be inhibited, and stabilization at the time of process continuous operation and reductions of operation costs associated therewith and facility maintenance costs can also be expected.

### MODE FOR CARRYING OUT INVENTION

Although embodiments of the present invention are hereunder described in detail, it should be construed that the present invention is by no means limited to the following embodiments and can be variously modified and carried out within the scope of the gist thereof

### <Production of Furfural>

In the production method of furfural according to the present invention, a composition containing furfural as a raw material contains furfural as a main component, and a concentration of furfural in the composition is not particularly limited. The concentration of furfural in the composition is preferably from 87.0% by mass or more, more preferably from 90.0% by mass or more, and especially preferably from 91.0% by mass or more.

On the other hand, the concentration of furfural in the composition is preferably from 99.0% by mass or less, more preferably from 98.5% by mass or less, and still more preferably from 98.0% by mass or less.

As the concentration of furfural in the composition becomes lower, there is a tendency that the concentration of a high-boiling component becomes higher, or the purity of furfural after purification by distillation is decreased. Conversely, as this concentration becomes higher, high-degree pretreatment purification facilities become necessary, and therefore, there is a tendency that facility costs or raw material costs needed for the furfural production are worsened.

In the production method of furfural according to the present invention, the composition containing furfural as a raw material to be used can be, for example, obtained from crude furfural. As the crude furfural, one obtained by a method in which a plant (nonedible biomass resource) containing a hemicellulose component, such as corncob, bagasse, sawdust of wood, etc., or the like is heated in the presence of an acid, such as dilute sulfuric acid, etc., to generate furfural and water, and a mixture containing furfural and water as thus generated is then subjected to a dehydration treatment is general. However, the crude furfural is not always limited to one obtained by this method, but a mixture containing furfural or the like may also be used as the crude furfural.

In the production method of furfural according to the present invention, it is preferred to include a step of obtaining a composition containing furfural from the crude furfural in advance prior to producing furfural from the composition containing furfural as a raw material.

In addition, in the foregoing step, it is preferred that before distilling the composition containing furfural by a distillation column, a compound having a higher boiling point than furfural in the crude furfural obtained after bringing the crude furfural into contact with an anion exchange resin and/or a basic compound is concentrated to obtain the composition containing furfural.

In addition, it is preferred that after bringing the crude furfural into contact with an anion exchange resin and/or a basic compound, a component having a lower boiling point than furfural is further removed by means of distillation By providing this step, a concentration of an acid component in the composition containing furfural as a raw material is decreased, whereby the polymerization is readily inhibited

Although the above-described anion exchange resin is not particularly limited, from the viewpoints of bringing an appropriate basicity and easily achieving regeneration, it is preferable that the anion exchange resin is a weakly basic anion exchange resin. Specifically, examples thereof include a weakly basic anion exchange resin, such as an acrylic type, a styrene-based polyamine type, etc.; and a strongly basic anion exchange resin having a trimethylammonium group or a dimethylethanolammonium group, or the like.

Although the above-described basic compound is not particularly limited, examples thereof include a basic inorganic compound, a basic organic compound, and the like.

Examples of the basic inorganic compound include a hydroxide of an alkali metal, such as lithium hydroxide, sodium hydroxide, potassium hydroxide, etc., a hydroxide of an alkaline earth metal, such as barium hydroxide, calcium hydroxide, etc.; and a carbonate, such as sodium carbonate, potassium carbonate, sodium bicarbonate, etc.

Specific examples of the basic organic compound include methylamine, etheramine, ethylamine, trimethylamine, triethylamine, tributylamine, triethanolamine, N,N-diisopropylethylamine, piperidine, piperazine, morpholine, quinuclidine, 1,4-diazabicyclooctane, pyridine, 4-dimethylaminopyridine, ethylenediamine, tetramethylethylenediamine, hexamethylenediamine, aniline, catecholamine, phenethylamine, 1,8-bis(dimethylamino)naphthalene (proton sponge), and the like.

Although an amount of the anion exchange resin and/or the basic compound to be brought into contact with the crude furfural is not particularly limited, it is preferably from 0.005 to 1% by mass, more preferably from 0.01 to 0.5% by mass, and still more preferably from 0.03 to 0.3% by mass relative to the amount of the crude furfural.

A mode of the contact between the anion exchange resin and/or the basic compound and the crude furfural is not particularly limited, and any means of a fixed bed flow type or a batch type, or the like may be taken.

Although a contact temperature in the fixed bed flow type is not particularly limited, it is preferably in the range of from 10° C. to 90° C., more preferably in the range of from 15° C. to 70° C., and especially preferably in the range of from 20° C. to 60° C. Although a retention time is not particularly limited, it is, for example, from 0.05 hours to 10 hours, preferably from 0.1 hours to 5 hours, and more preferably from 0.5 hours to 2 hours.

Although a contact temperature in the batch type is not particularly limited, it is preferably in the range of from 10° C. to 90° C., more preferably in the range of from 15° C. to 70° C., and especially preferably in the range of from 20° C. to 50° C. Though a contact time is not particularly limited, it is, for example, 0.5 hours to 20 hours, preferably 0.5 hours to 10 hours, and more preferably 1 hour to 5 hours.

It is preferred that as described above, the crude furfural is brought into contact with the anion exchange resin and/or the basic compound and then distilled using a distillation column, and a compound having a higher boiling point than furfural is concentrated, whereby the composition containing furfural as a raw material, which is used in the production method of furfural according to the present invention, is obtained from the column bottom. The distillation column to be used on that occasion is not particularly limited, and any of a batch type or continuous distillation may be used. However, continuous distillation in which it is easy to control the concentration of the furfural dimer or furancarboxylic acid is preferred. As for the mode, any of a plate column using a sieve tray or bubble cap tray, etc. or a packed column using regular packings or irregular packings may be adopted.

Although a distillation condition in this distillation is not particularly limited, the number of theoretical plate is in the range of from 1 to 50 plates, preferably from 3 to 40 plates, and more preferably 5 to 30 plates. Although a feed temperature of the crude furfural into the distillation column is not particularly limited, it is from −20 to 120° C., preferably from 0 to 100° C., and more preferably from 10 to 80° C. Although a column top pressure within the distillation column is not particularly, it is from 0.12 to 28.2 kPa, preferably from 0.5 to 20.5 kPa, and more preferably from 0.8 to 15.5 kPa.

As the compound having a higher boiling point than furfural, in general, compounds having a boiling point higher by at least 5° C. than the boiling point of furfural at atmospheric pressure are exemplified. Examples thereof include compounds, such as furfuryl alcohol having a boiling point of 170° C., 2-furancarbonyl chloride having a boiling point of 173 to 174° C., 2-acetylfuran having a boiling point of 173° C., 5-methylfurfural having a boiling point of 187° C., furyl methyl ketone, etc., relative to furfural having a boiling point of 162° C. at atmospheric pressure.

Although a proportion of the compound having a higher boiling point higher than furfural to be concentrated, is not particularly limited, it is typically from 30% by mass or more, preferably from 50% by mass or more, more preferably from 75% by mass or more, and still more preferably from 90% by mass or more on a basis of the total mass (100% by mass) of compounds having a high boiling point contained in the crude furfural

The production method of furfural according to the present invention is characterized in that in distilling the composition containing furfural by a distillation column to obtain furfural, a concentration of the furfural dimer in a column bottom liquid of the distillation column is 20 ppm by mass to 5,000 ppm by mass. Then, in the production method of furfural according to the present invention, it is preferred to control the concentration of the furfural dimer in the column bottom liquid of the distillation column to the range of from 20 to 5,000 ppm by mass.

This furfural dimer is a dimer of furfural, and specifically, (5-(2-furanylcarbonyl)-2-furancarboxyaldehyde and di-2-furylethanedione are preferred as the furfural dimer in the production method of furfural according to the present invention.

When the concentration of the furfural dimer exceeds the upper limit of the foregoing range, there is a concern that a by-produced solid matter is remarkably formed, whereas when it is less than the lower limit of the foregoing range, there is a concern that the generation of a side-reaction to be caused due to a minute amount of a metal component existing in the distillation column bottom (one resulting from elution of a metal existing in the surface of the distillation column or pipe, etc.) is hardly inhibited, whereby the efficiency of the production method of furfural according to the present invention is worsened.

In the production method of furfural according to the present invention, a concentration of the furfural dimer in the column bottom liquid of the distillation column for obtaining furfural through distillation of the composition containing furfural as a raw material is from 20 ppm by mass or more, preferably from 100 ppm by mass or more, more preferably from 200 ppm by mass or more, and especially preferably from 1,000 ppm by mass or more.

On the other hand, this concentration is 5,000 ppm by mass or less, preferably from 4,500 ppm by mass or less, more preferably from 4,000 ppm by mass or less, and still more preferably from 3,500 ppm by mass or less.

In order that the concentration of the furfural dimer in the column bottom liquid of the distillation column for distilling the composition containing furfural to obtain furfural to be less than the lower limit of the foregoing concentration range, the column bottom temperature of the distillation column must be excessively decreased, or the retention time must be excessively shortened, and the operation becomes inefficient from the standpoint of operating the distillation column. Therefore, such is not preferred. In addition, as this concentration becomes higher, there is a tendency that it becomes difficult to inhibit the formation amount of a by-produced solid matter.

Although a distinct reason why by controlling the concentration of the furfural dimer of the column bottom liquid of the distillation column for distilling the composition containing furfural as a raw material to obtain furfural within the foregoing range, it becomes possible to inhibit the formation of a by-produced solid matter is not elucidated yet, the following reasons may be conjectured. Namely, it may be considered that when the furfural dimer is present, as compared with easiness of the formation of a polymer obtained through polymerization between two furfurals, a low polymer between this furfural dimer and furfural is more easily formed. It may be considered that particularly, this replies upon the temperature condition in the column bottom of the distillation column, and the generation of polymerization readily occurs according to the temperature condition. Then, as a result, it is considered that when the concentration of this furfural excessively increases, a polymerization reaction is caused between the furfural dimer and furfural and between the two furfural dimers, resulting in the formation of a solid matter.

Although a method of controlling a concentration of the furfural dimer in the column bottom liquid of the distillation column for distilling the composition containing furfural as a raw material to obtain furfural is not particularly limited, examples thereof include a method of controlling a concentration factor; a method of controlling a column bottom temperature of the distillation column in order to inhibit the formation of a furfural dimer within the distillation column; a method of controlling a radical source, such as oxygen, a peroxide, light, an organic radical, etc.; and a method of adjusting an acidity of the column bottom liquid or the composition containing furfural as a raw material.

In addition, there are also exemplified a method of controlling a concentration of the furfural dimer in the composition containing furfural as a raw material through distillation; a method of achieving dilution with furfural having a high purity; a method of adding a furfural dimer; and the like. Above all, a method of controlling a column bottom temperature of the distillation column and a method of adjusting an acidity of the column bottom liquid are preferred.

In the production method of furfural according to the present invention, the column bottom temperature of the distillation column for distilling the composition containing furfural as a raw material to obtain furfural is preferably from 60 to 180° C., more preferably from 70 to 160° C., and still more preferably from 80 to 140° C. When the temperature is too low, the column top pressure must be excessively decreased, so that there is a concern that the continuation of operation of the distillation column becomes difficult from the standpoint of facilities or costs. Conversely, when this temperature is too high, there is a tendency that the formation of a solid matter increases.

In the production method of furfural according to the present invention, the acidity of the column bottom liquid of the distillation column for distilling the composition containing furfural as a raw material to obtain furfural is preferably from 10 mg-KOH/g or less, more preferably from 9 mg-KOH/g or less, and especially preferably from 8.5 mg-KOH/g or less in terms of an acid value.

When this value is excessively high, there is a tendency that the formation of a solid matter increases. Although a method of adjusting this acid value is not particularly limited, it becomes possible to achieve the adjustment by combining methods, such as a method of adding a basic substance to the column bottom liquid, a method of treating the crude furfural as a raw material with a base, a method of decomposing the acidic substance by means of decarboxylation by heating or the like, etc.

As a method of measuring the acid value of the column bottom liquid during the distillation, there is exemplified a method of sampling the column bottom liquid and titrating it with a potassium hydroxide solution or the like. In addition, a measured value with a commercially available pH meter (online pH meter) which is capable of automatically performing continuous measurement without sampling, or the like may also be used

In the production method of furfural according to the present invention, from the viewpoint that when a concentration of furancarboxylic acid in the column bottom liquid of the distillation column for distilling the composition containing furfural as a raw material to obtain furfural is controlled, the formation of a solid matter can be further reduced, it is preferred that this concentration of furancarboxylic acid in the column bottom liquid of the distillation column is from 50 to 8,000 ppm by mass.

In addition, it is more preferred to control the concentration of furancarboxylic acid in the column bottom liquid of the distillation column to the range of from 50 to 8,000 ppm by mass. This concentration range is preferably from 50 ppm by mass or more, more preferably from 200 ppm by mass or more, and especially preferably from 500 ppm by mass or more. On the other hand, this concentration is preferably from 8,000 ppm by mass or less, more preferably from 6,000 ppm by mass or less, and still more preferably from 5,000 ppm by mass or less

As this concentration becomes lower, the concentration of oxygen in the distillation column must be excessively decreased, and from the viewpoint of operation condition or production costs, the production must be achieved under an inefficient condition, and hence, such is not preferred. On the other hand, as this concentration becomes higher, there is a tendency that the formation amount of a solid matter increases.

Although a distinct reason why by controlling the concentration of furancarboxylic acid in the column bottom liquid of the distillation column for distilling the composition containing furfural as a raw material to obtain furfural, it becomes possible to inhibit the formation of a solid matter is not elucidated yet, the following reasons may be conjectured. Namely, it may be conjectured that an excessive increase of the furancarboxylic acid contributes to an increase of the acid value of the column bottom liquid of the distillation column, and as a result, polymerization of the above-described furfural dimer and furfural is readily advanced. For that reason, by controlling the concentrations of both of the furfural dimer and furancarboxylic acid, a more formation inhibiting effect of a solid matter can be expected. In addition, it may also be conjectured that the furancarboxylic acid reacts with a minute amount of oxygen within the distillation column to form a peroxide, thereby causing the polymerization. Thus, it may be considered that there is such an effect for inhibiting a by-produced solid matter other than the furfural dimer.

In accordance with the production method of furfural according to the present invention, the concentration of a component other than the furfural dimer and having a higher boiling point than furfural at the time of distillation and concentration can be decreased, too, and therefore, the formation of a solid matter can be inhibited. The concentration of this component is preferably from 0.3% by mass or more, more preferably from 1% by mass or more, and especially preferably from 3% by mass or more relative to the furfural-containing liquid in terms of the concentration at the time of distillation and concentration.

On the other hand, this concentration is preferably from 17.5% by mass or less, more preferably from 16% by mass or less, and still more preferably from 15% by mass or less. In order to make this concentration lower than the foregoing range, the degree of concentration of furfural must be excessively decreased, and furfural cannot be recovered from the high-boiling component, and hence, such is not economically preferred. In addition, in the case where this concentration is too high, the formation amount of a solid matter increases.

In the production method of furfural according to the present invention, although a treatment mode of the distillation column for distilling the composition containing furfural as a raw material to obtain furfural may be any of a batch type or continuous distillation, continuous distillation is preferred. As for the distillation mode, any of a plate column using a sieve tray or bubble cap tray, etc. or a packed column using regular packings or irregular packings may be used. Although a distillation condition is not particularly limited, the number of theoretical plate is in the range of from 1 to 50 plates, preferably from 3 to 30 plates, and more preferably 5 to 20 plates. A column top pressure within the distillation column is from 0.12 to 28.2 kPa, preferably from 0.5 to 20.5 kPa, and more preferably from 0.8 to 15.5 kPa.

In the production method of furfural according to the present invention, as for a method of distilling the composition containing furfural by the distillation column and measuring the concentration of the furfural dimer or the concentration of furancarboxylic acid in the column bottom liquid of the distillation column, the column bottom liquid of the distillation column may be directly extracted, followed by measuring the concentration with an analyzer, or the liquid in a line during sending the column bottom liquid to a subsequent step may be extracted, followed by measuring the concentration with an analyzer. The analysis of the concentration may be any of continuous online analysis or intermittent step analysis. It is preferred to perform monitoring so as to fall within the above-described numerical value range of the concentration of the furfural dimer and/or numerical value range of the concentration of furancarboxylic acid, with monitoring the measured concentration.

### <Production of Furan>

Furan can be produced by subjecting the furfural obtained in the above-described production method of furfural according to the present invention to a decarbonylation reaction in the presence of a catalyst. Prior to supplying the furfural as a raw material for the production of furan, this furfural may be further subjected to a purification treatment, such as distillation, etc., in advance.

The furan formed by the production method of furan according to the present invention is separated from carbon monoxide and by-products which are by-produced by the reaction, unreacted furfural, nitrogen, hydrogen, and the like and then purified by an operation, such as absorption, distillation, etc. It is also possible for the separated carbon monoxide to be recycled as a carrier gas for the decarbonylation reaction, effectively used for other applications, or burnt and subjected to heat recovery.

Although the decarbonylation reaction may be any of a liquid phase or gas phase reaction, in the production method of furan according to the present invention, the gas phase reaction is preferred. The reaction mode of the decarbonylation reaction is not particularly prescribed, and it can be carried out by any of a batch reaction or a continuous flow reaction. However, it is preferred to use a continuous flow reaction mode from the industrial standpoint

In the case of a gas phase flow reaction, typically, a furfural gas containing, as a main component, furfural is continuously fed as a raw material into a tubular reactor filled with a catalyst and passed through the catalyst within the reactor to advance the reaction, thereby obtaining furan. In the case of producing furan as a production process in which the above-described production method of furfural according to the present invention is consistently integrated with the production method of furan, it is preferred that prior to supplying the furfural as the raw material of furan into the reactor, it is previously gasified in a vaporizer provided before the reactor. Although this gasification method is not particularly limited, examples thereof include a method in which the furfural in a liquid state is subjected to gas bubbling with hydrogen, an inert gas, or the like; a method of gasifying the furfural by means of spray gasification; and the like.

In the production method of furan according to the present invention, it is preferable that a moisture concentration in furfural to be supplied for the decarbonylation reaction is 10 ppm by mass or more and 1% by mass or less; it is more preferable that 15 ppm by mass or more and 1,000 ppm by mass or less; and it is still more preferable that 20 ppm by mass or more and 500 ppm by mass or less. When the moisture concentration is too high, there is a tendency that the yield is lowered, whereas when it is too low, there is a tendency that the raw material purification load becomes large.

In addition, in this decarbonylation reaction, it is suitable to allow hydrogen to coexist as a reaction initiator.

In the production method of furan according to the present invention, although a feed amount of furfural to be fed into the reactor is not particularly limited, it is typically 0.0001 mol/h or more and 50,000 mol/h or less; it is preferable that 0.001 mol/h or more and 10,000 mol/h or less; and it is more preferable that 0.01 mol/h or more and 5,000 mol/h or less per mol of a noble metal bearing the catalytic activity.

In the production method of furan according to the present invention, in the case where the reaction mode for performing the decarbonylation reaction is a gas phase flow reaction, although a retention time thereof is not particularly limited, it is typically 0.001 seconds or more and 10 seconds or less; it is preferable that 0.01 seconds or more and 5 seconds or less; it is more preferable that 0.05 seconds or more and 2 seconds or less; and it is especially preferable that 0.1 seconds or more and 1 second or less.

Although a reaction temperature is not particularly limited, in general, it is preferable that 170° C. or higher and 450° C. or lower; it is more preferable that 180° C. or higher and 380° C. or lower; and it is still more preferable that 200° C. or higher and 340° C. or lower; and it is especially preferable that 230° C. or higher and 300° C. or lower. When the reaction temperature is too low, the furfural compound is hard to be sufficiently converted, whereas when the reaction temperature is too high, the formed furan compound causes a successive reaction, and as a result, there is a tendency that the yield of the furan compound is lowered.

Although a reaction pressure is not particularly limited, it is typically 0.01 MPa or more and 3 MPa or less; it is preferable that 0.05 MPa or more and 2 MPa or less; and it is more preferable that 0.1 MPa or more and 1 MPa or less, in terms of an absolute pressure.

Although a catalyst that is used for the decarbonylation reaction is not particularly limited, a solid catalyst is preferably used. As a catalyst metal of the solid catalyst, at least one metal selected from transition metal elements belonging to the Groups 8 to 10 of the Periodic Table is suitably used. As the transition metal elements belonging to the Groups 8 to 10 of the Periodic Table, Ni, Ru, Ir, Pd, and Pt are preferred, Ru, Ir, Pd, and Pt are more preferred; Pd and Pt are still more preferred. Above all, Pd whose selectivity for conversion of from furfural into furan is extremely high is especially preferred.

Although the kind of a carrier is not particularly limited, carriers of single metal oxides, such as Al₂O₃, SiO₂, TiO₂, ZrO₂, MgO, etc., and complex metal oxides thereof, porous oxides, such as zeolite, etc., and active carbon, can be used. In order to improve the performance of the catalyst, such a supported metal catalyst can contain a modification assistant. Examples of the modification assistant include the Group 1 metals and ions thereof, the Group 2 metals and ions thereof, the Group 4 metals and ions thereof, and the Group 6 metals and ions thereof, wherein the Group 1 metals and ions thereof are preferred.

The obtained furan compound is useful as a variety of resin raw materials and additives and also useful as intermediates for derivative synthesis. For example, the furan compound can be converted into tetrahydrofuran through a hydrogenation reaction with using a catalyst. Although a production method of tetrahydrofuran is not particularly limited, it is preferred to produce tetrahydrofuran from furan through a hydrogenation reaction with using a catalyst having an element belonging to the Groups 8 to 10 supported on a carrier, such as active carbon, etc. In addition, the furan compound can also be converted into 1,4-butanediol or γ-butyrolactone through a combination with hydration or the like.

### EXAMPLES

Although the present invention is hereunder described in more detail with reference to Examples, it should be construed that the present invention is not limited by the following Examples so long as the gist of the present invention is not deviated.

In the following Examples, the analysis of moisture was performed by the Karl Fischer method (measurement apparatus: CA-21, manufactured by Mitsubishi Chemical Corporation). The analysis of each of furfural, a furfural dimer, and furancarboxylic acid was performed by means of gas chromatography, and using dioxane as an internal standard substance of the gas chromatography, a concentration of each of the components was calculated from a separately prepared calibration curve.

In addition, a high-boiling material that could not be detected by the gas chromatography (hereinafter abbreviated as “GC-outside HB”) was calculated by subtracting a total value of the concentrations of compounds obtained by the gas chromatography by the internal standard method and a moisture value from 100.

### Production Example 1

### Production of Composition Containing Furfural

A glass-made chromatographic tube having a capacity of 100 cc and equipped with a jacket capable of being heated by circulating warm water was filled with 70 cc of an anion exchange resin (“DIAION” (a registered trademark), manufactured by Mitsubishi Chemical Corporation, model name: WA20), and furfural (purity: 98.7% by mass), manufactured by Kanematsu Chemicals Corporation was circulated at a rate of 140 cc/h into this glass-made chromatographic tube. On that occasion, a contact temperature between the anion exchange resin and furfural was 40° C., and a pressure was atmospheric pressure.

With using an Oldershaw distillation column having a column diameter of 35 mm and the number of theoretical plate of 5 plates, 1000.0 g of the obtained furfural was distilled at a column top pressure of 13.3 kPa and a column bottom temperature of 102° C. An oil bath was used as a heat source of the distillation, and a temperature of the oil bath was set to 120° C. A distillate was extracted successively from an initial distillate containing a lot of a light-boiling component, and when the distillation reached a proportion of 90% by mass relative to the furfural in the column bottom liquid of the distillation column, the distillation was terminated. At that time, the composition and acid value of a solution remained in the pot are those shown in the following Table 1.

TABLE 1

| Component name | Concentration [% by mass] |
|---|---|
| Furfural | 91.3 |
| Furancarboxylic acid | 0.01 |
| Furfural dimer | 0.02 |
| GC-outside HB | 0.47 |
| Acid value | 1.1 mg-KOH/g |

### Example 1

### Production of Furfural

In a 100-cc glass-made flask having a glass-made condenser for distillation installed therein, 40.0 g of the pot residue of Production Example 1 was charged and subjected to simple distillation in an atmosphere at a pressure of 13.3 kPa, a liquid temperature within the flask of 100° C., and an oxygen concentration 20 ppm by volume.

At the point of time when 32.1 g of furfural was obtained as the distillate, 2.0 g of the pot residue within the flask was sampled, and then, the distillation was terminated. An amount of the liquid remained within the flask was 5.5 g. In the sampled pot residue, the concentration of furfural dimer was 1,748 ppm by mass; the concentration of furancarboxylic acid was 1,810 ppm by mass; the GC-outside HB was 3.1% by mass; and the acid value was 3.7 mg-KOH/g. At that time, a solid matter was not observed in the still pot.

### Example 2

The same procedures as in Example 1 were all carried out, except for obtaining 35.7 g of the distillate. At the point of time when the concentration ratio was 2 times, 5 times, and 10 times, respectively, 0.1 g of every liquid was extracted, and the distillation was performed with confirming that the concentration of furfural dimer was 5,000 ppm by mass or less, and the concentration of furancarboxylic acid was 8,000 ppm by mass or less. The amount of the liquid after distillation after the concentration of 10 times was 1.5 g, and in the sampled pot residue, the concentration of furfural dimer was 2,821 ppm by mass; the concentration of furancarboxylic acid was 3,312 ppm by mass; the GC-outside HB was 6.4% by mass; and the acid value was 8.1 mg-KOH/g. At that time, a solid matter was not observed in the still pot.

### Example 3

The same procedures as in Example 2 were all carried out, except for controlling the oxygen concentration to 1,000 ppm by volume and the temperature within the flask to 120° C. The amount of the liquid after distillation was 1.7 g, and in the sampled pot residue, the concentration of furfural dimer was 2,811 ppm by mass; the concentration of furancarboxylic acid was 4,839 ppm by mass; the GC-outside HB was 8.2% by mass; and the acid value was 8.4 mg-KOH/g. At that time, a solid matter was not observed in the still pot.

### Example 4

The same procedures as in Example 2 were all carried out, except for controlling the temperature within the flask to 180° C. The amount of the liquid after distillation was 1.8 g, and in the sampled pot residue, the concentration of furfural dimer was 3,001 ppm by mass; the concentration of furancarboxylic acid was 3,405 ppm by mass; the GC-outside HB was 17.4% by mass; and the acid value was 8.2 mg-KOH/g. At that time, a minute amount (0.3 mg) of a solid matter was observed in the still pot.

### Example 5

The same procedures as in Example 2 were all carried out, except for controlling the oxygen concentration to 1,000 ppm by volume and the temperature within the flask to 180° C. and adding 1,000 ppm by mass of trioctylamine. The amount of the liquid after distillation was 1.7 g, and in the sampled pot residue, the concentration of furfural dimer was 3,134 ppm by mass; the concentration of furancarboxylic acid was 4,529 ppm by mass; the GC-outside FIB was 14.1% by mass; and the acid value was 7.9 mg-KOH/g. At that time, a solid matter was not observed in the still pot.

### Comparative Example 1

The same procedures as in Example 1 were all carried out, except for obtaining 36.5 g of the distillate. The amount of the liquid after distillation was 1.5 g, and in the sampled pot residue, the concentration of furfural dimer was 6,240 ppm by mass; the concentration of furancarboxylic acid was 8,604 ppm by mass; and the GC-outside HB was 14.1% by mass. At that time, 1,800 mg of a solid matter was observed in the still pot.

TABLE 2

| | Concentration | | | | | | | | |
|---|---|---|---|---|---|---|---|---|---|
| | of | Concentration | | | Oxygen | | Concen- | | Amount of |
| | furancarboxylic | of | GC-outside | | concentration | Distillation | tration | | formed solid |
| | acid | furfural dimer | HB | Acid value | (ppm by | temperature | ratio | | matter |
| | (ppm) | (ppm) | (% by mass) | (mg-KOH/g) | volume) | (° C.) | (times) | Additive | (mg) |
| Example 1 | 1810 | 1748 | 3.1 | 3.7 | 20 | 100 | 5 | No | No |
| Example 2 | 3312 | 2821 | 6.4 | 8.1 | 20 | 100 | 10 | No | No |
| Example 3 | 4839 | 2811 | 8.2 | 8.4 | 1000 | 120 | 10 | No | No |
| Example 4 | 3405 | 3001 | 17.4 | 8.2 | 20 | 180 | 10 | No | 0.3 |
| Example 5 | 4529 | 3134 | 14.1 | 7.9 | 1000 | 180 | 10 | Trioctylamine | No |
| Comparative | 8604 | 6240 | 17.2 | Unmeasurable | 20 | 100 | 20 | No | 1800 |
| Example 1 | | | | | | | | | |

### Example 6

### Heating of Composition Containing Furfural

In a glass-made 50-mL Schlenk flask, furfural having a purity of 98.5% was charged, and reagents were added such that the amounts of furyl (furfural dimer) and furancarboxylic acid were 500 ppm by mass and 500 ppm by mass, respectively, to prepare a composition containing furfural, which was then heated for 5 hours in an atmosphere at a liquid temperature within the flask of 180° C. and an oxygen concentration of 20 ppm by volume. At that time, the liquid level of the heat medium was made taller than the furfural liquid level. As a result of measuring the amount of a solid matter formed after heating, it was found to be 4.1 mg.

### Example 7

The same procedures as in Example 6 were all carried out, except for adjusting the concentration of furfural dimer in the composition containing furfural to be 1,000 ppm by mass. As a result of measuring the amount of a solid matter formed after heating, it was found to be 2.8 mg.

### Example 8

The same procedures as in Example 6 were all carried out, except for adjusting the concentration of furfural dimer in the composition containing furfural to be 3,000 ppm by mass. As a result of measuring the amount of a solid matter formed after heating, it was found to be 1.9 mg.

### Example 9

The same procedures as in Example 6 were all carried out, except for adjusting the concentration of furfural dimer in the composition containing furfural to be 4,500 ppm by mass. As a result of measuring the amount of a solid matter formed after heating, it was found to be 0.9 mg.

### Comparative Example 2

The same procedures as in Example 6 were all carried out, except for adjusting the concentration of furfural dimer in the composition containing furfural to be 6,000 ppm by mass. As a result of measuring the amount of a solid matter formed after heating, it was found to be 14.5 mg.

### Comparative Example 3

The same procedures as in Example 6 were all carried out, except for adjusting the concentration of furfural dimer in the composition containing furfural to be 7,500 ppm by mass. As a result of measuring the amount of a solid matter formed after heating, it was found to be 16.0 mg.

### Example 10

The same procedures as in Example 6 were all carried out, except for adjusting the concentration of furfural dimer in the composition containing furfural to be 1,000 ppm by mass and the concentration of furancarboxylic acid to be 3,000 ppm by mass. As a result of measuring the amount of a solid matter formed after heating, it was found to be 3.3 mg.

### Example 11

The same procedures as in Example 7 were all carried out, except for adjusting the concentration of furancarboxylic acid in the composition containing furfural to be 7,000 ppm by mass. As a result of measuring the amount of a solid matter formed after heating, it was found to be 1.5 mg.

### Example 12

The same procedures as in Example 11 were all carried out, except for adding 3,000 ppm by mass of aminodecane in the composition containing furfural. As a result of measuring the amount of a solid matter formed after heating, it was found to be 1.4 mg.

### Comparative Example 4

The same procedures as in Example 7 were all carried out, except for adjusting the concentration of furancarboxylic acid in the composition containing furfural to be 9,000 ppm by mass. As a result of measuring the amount of a solid matter formed after heating, it was found to be 13.4 mg.

### Comparative Example 5

The same procedures as in Example 7 were all carried out, except for adjusting the concentration of furancarboxylic acid in the composition containing furfural to be 11,000 ppm by mass. As a result of measuring the amount of a solid matter formed after heating, it was found to be 34.1 mg.

### Example 13

The same procedures as in Example 6 were all carried out, except for adjusting the concentration of furfural dimer in the composition containing furfural to be 3,000 ppm by mass and the concentration of furancarboxylic acid to be 3,000 ppm by mass. As a result of measuring the amount of a solid matter formed after heating, it was found to be 0.2 mg.

### Example 14

The same procedures as in Example 6 were all carried out, except for adjusting the concentration of furfural dimer in the composition containing furfural to be 4,500 ppm by mass and the concentration of furancarboxylic acid to be 7,000 ppm by mass. As a result of measuring the amount of a solid matter formed after heating, it was found to be 0.3 mg.

### Comparative Example 6

The same procedures as in Example 6 were all carried out, except for adjusting the concentration of furfural dimer in the composition containing furfural to be 6,000 ppm by mass and the concentration of furancarboxylic acid to be 9,000 ppm by mass. As a result of measuring the amount of a solid matter formed after heating, it was found to be 15.1 mg.

### Comparative Example 7

The same procedures as in Example 6 were all carried out, except for adjusting the concentration of furfural dimer in the composition containing furfural to be 7,500 ppm by mass. As a result of measuring the amount of a solid matter formed after heating, it was found to be 21.2 mg.

TABLE 3

| | Concentration | | Concentration | | | | | Amount of |
|---|---|---|---|---|---|---|---|---|
| | of | Concentration of | of | GC-outside HB | Oxygen | Heating | | solid matter |
| | furfural | furancarboxylic acid | furfural dimer | after heating | concentration | temperature | | formed |
| | (% by mass) | (ppm by mass) | (ppm by mass) | (% by mass) | (ppm by volume) | (° C.) | Additive | (mg) |
| Example 6 | 98.5 | 500 | 500 | 0.11 | 20 | 180 | No | 4.1 |
| Example 7 | 98.5 | 500 | 1000 | 0.10 | 20 | 180 | No | 2.8 |
| Example 8 | 98.3 | 500 | 3000 | 0.12 | 20 | 180 | No | 1.9 |
| Example 9 | 98.2 | 500 | 4500 | 0.13 | 20 | 180 | No | 0.9 |
| Comparative | 98.3 | 500 | 6000 | 0.11 | 20 | 180 | No | 14.5 |
| Example 2 | | | | | | | | |
| Comparative | 98.1 | 500 | 7500 | 0.11 | 20 | 180 | No | 16 |
| Example 3 | | | | | | | | |
| Example 10 | 98.3 | 3000 | 1000 | 0.11 | 20 | 180 | No | 3.3 |
| Example 11 | 98.2 | 7000 | 1000 | 0.10 | 20 | 180 | No | 1.5 |
| Example 12 | 98 | 7000 | 1000 | 0.10 | 20 | 180 | Aminodecane | 1.4 |
| Comparative | 98 | 9000 | 1000 | 0.15 | 20 | 180 | No | 13.4 |
| Example 4 | | | | | | | | |
| Comparative | 97.8 | 11000 | 1000 | 0.14 | 20 | 180 | No | 34.1 |
| Example 5 | | | | | | | | |
| Example 13 | 98 | 3000 | 3000 | 0.12 | 20 | 180 | No | 0.2 |
| Example 14 | 97.8 | 7000 | 4500 | 0.10 | 20 | 180 | No | 0.3 |
| Comparative | 97.5 | 9000 | 6000 | 0.20 | 20 | 180 | No | 15.1 |
| Example 6 | | | | | | | | |
| Comparative | 98.2 | 500 | 7500 | 0.13 | 1000 | 180 | No | 21.2 |
| Example 7 | | | | | | | | |

### Example 15

The same procedures as in Example 6 were all carried out, except for using, as the composition containing furfural, the furfural of Production Example 1; adjusting the concentration of furfural dimer to be 200 ppm by mass and the concentration of furancarboxylic acid to 100 ppm by mass with reagents; and making the liquid level of the heat medium equal to the liquid level of the furfural solution. As a result of measuring the amount of a solid matter formed after heating, it was found to be 3.3 mg.

### Example 16

The same procedures as in Example 15 were all carried out, except for adjusting the concentration of furfural dimer to be 4,500 ppm by mass and the concentration of furancarboxylic acid to be 500 ppm by mass. As a result of measuring the amount of a solid matter formed after heating, it was found to be 2.1 mg.

### Comparative Example 8

The same procedures as in Example 16 were all carried out, except for adjusting the concentration of furfural dimer to be 6,000 ppm by mass. As a result of measuring the amount of a solid matter formed after heating, it was found to be 9.8 mg.

### Example 17

The same procedures as in Example 15 were all carried out, except for adjusting the concentration of furfural dimer to be 4,500 ppm by mass and the concentration of furancarboxylic acid to 7,000 ppm by mass. As a result of measuring the amount of a solid matter formed after heating, it was found to be 1.3 mg.

### Comparative Example 9

The same procedures as in Example 15 were all carried out, except for adjusting the concentration of furfural dimer to be 1,000 ppm by mass and the concentration of furancarboxylic acid to be 9,000 ppm by mass. As a result of measuring the amount of a solid matter formed after heating, it was found to be 31.0 mg.

### Comparative Example 10

The same procedures as in Comparative Example 9 were all carried out, except for adjusting the concentration of furancarboxylic acid to be 11,000 ppm by mass. As a result of measuring the amount of a solid matter formed after heating, it was found to be 46.5 mg.

### Comparative Example 11

The same procedures as in Comparative Example 10 were all carried out, except for adjusting the liquid temperature within the flask to be 170° C. As a result of measuring the amount of a solid matter formed after heating, it was found to be 20.6 mg.

### Comparative Example 12

The same procedures as in Example 15 were all carried out, except for adjusting the concentration of furfural dimer to be 6,000 ppm by mass and the concentration of furancarboxylic acid to be 9,000 ppm by mass. As a result of measuring the amount of a solid matter formed after heating, it was found to be 28.0 mg.

TABLE 4

| | Concentration | | Concentration | | | | | |
|---|---|---|---|---|---|---|---|---|
| | of | Concentration of | of | GC-outside HB | Oxygen | Heating | | Amount of solid |
| | furfural | furancarboxylic acid | furfural dimer | after heating | concentration | temperature | | matter formed |
| | (% by mass) | (ppm by mass) | (ppm by mass) | (% by mass) | (ppm by volume) | (° C.) | Additive | (mg) |
| Example 15 | 91.3 | 100 | 200 | 1.90 | 20 | 180 | No | 3.3 |
| Example 16 | 87.9 | 500 | 4500 | 4.50 | 20 | 180 | No | 2.1 |
| Comparative | 90.0 | 500 | 6000 | 2.16 | 20 | 180 | No | 9.8 |
| Example 8 | | | | | | | | |
| Example 17 | 90.4 | 7000 | 4500 | 1.46 | 20 | 180 | No | 1.3 |
| Comparative | 89.1 | 9000 | 1000 | 2.99 | 20 | 180 | No | 31 |
| Example 9 | | | | | | | | |
| Comparative | 87.7 | 11000 | 1000 | 5.02 | 20 | 180 | No | 46.5 |
| Example 10 | | | | | | | | |
| Comparative | 87.7 | 11000 | 1000 | 6.51 | 20 | 170 | No | 20.6 |
| Example 11 | | | | | | | | |
| Comparative | 87.6 | 9000 | 6000 | 5.16 | 20 | 180 | No | 28 |
| Example 12 | | | | | | | | |

### Example 18

The same procedures as in Example 6 were all carried out, except for adding iron sulfate heptahydrate in an amount of 10 ppm by mass in terms of an iron atom concentration; and adjusting the concentration of furfural dimer to be 200 ppm by mass and the concentration of furancarboxylic acid to be not more than a detection limit. The amount of furan formed after heating was not more than a detection limit, and the formation amount of a component having a lower boiling point than furfural was 7 ppm by mass.

### Example 19

The same procedures as in Example 18 were all carried out, except for adding nickel chloride hexahydrate in an amount of 10 ppm by mass in terms of a nickel atom concentration. The amount of furan formed after heating was not more than a detection limit, and the formation amount of a component having a lower boiling point than furfural was 7 ppm by mass.

### Example 20

The same procedures as in Example 18 were all carried out, except for adjusting the concentration of furfural dimer to be not more than a detection limit and the concentration of furancarboxylic acid to be 200 ppm by mass. The amount of furan formed after heating was 37 ppm by mass, and the formation amount of a component having a lower boiling point than furfural was 44 ppm by mass.

### Comparative Example 13

The same procedures as in Example 18 were all carried out, except for adjusting the concentration of furfural dimer to be not more than a detection limit. The amount of furan formed after heating was 78 ppm by mass, and the formation amount of a component having a lower boiling point than furfural was 607 ppm by mass.

### Comparative Example 14

The same procedures as in Example 19 were all carried out, except for adjusting the concentration of furfural dimer to be not more than a detection limit. The amount of furan formed after heating was 71 ppm by mass, and the formation amount of a component having a lower boiling point than furfural was 545 ppm by mass.

TABLE 5

| | Concen- | | | | | | | | |
|---|---|---|---|---|---|---|---|---|---|
| | tration | Concentration | | | | | | | Formation |
| | of | of | Concentration | GC-outside | Oxygen | Heating | | Formation | amount of low- |
| | furfural | furancarboxylic | of | HB after | concentration | tem- | Concentration of | amount | boiling |
| | (% by | acid | furfural dimer | heating | (ppm by | perature | iron or Ni | of furan | component |
| | mass) | (ppm by mass) | (ppm by mass) | (% by mass) | volume) | (° C.) | (ppm by mass) | (ppm by mass) | (ppm by mass) |
| Example 18 | 98.9 | Not more than | 200 | 0.10 | 20 | 180 | Fe: 10 ppm | Not more than | 7 |
| | | detection limit | | | | | | detection limit | |
| Example 19 | 98.9 | Not more than | 200 | 0.10 | 20 | 180 | Ni: 10 ppm | Not more than | 7 |
| | | detection limit | | | | | | detection limit | |
| Example 20 | 98.9 | 200 | Not more than | 0.10 | 20 | 180 | Fe: 10 ppm | 37 | 44 |
| | | | detection limit | | | | | | |
| Comparative | 98.9 | Not more than | Not more than | 0.10 | 20 | 180 | Fe: 10 ppm | 78 | 607 |
| Example 13 | | detection limit | detection limit | | | | | | |
| Comparative | 98.9 | Not more than | Not more than | 0.10 | 20 | 180 | Ni: 10 ppm | 71 | 545 |
| Example 14 | | detection limit | detection limit | | | | | | |

### Example 21

To 300.3 g of furfural (purity: 98.7% by mass), manufactured by Kanematsu Chemicals Corporation as a raw material, 0.8 g of triethylamine, manufactured by Tokyo Chemical Industry Co., Ltd. was added, and using an Oldershaw distillation column having a column diameter of 35 mm and the number of theoretical plate of 10 plates, batch distillation was carried out at a column top pressure of 12 kPa and a column bottom temperature of 125° C. Heating was performed for about 2 hours, and then, the distillation was finished. At that time, a sum total of the distillation amounts extracted from the column top of the distillation column relative to the amount of the raw material furfural charged in the distillation column was 75.5% by mass. Into a 100-cc four-neck flask, 40.1 g of the column bottom liquid after finish of the distillation was transferred and subjected to distillation over 2 hours at a pressure of the column top of 12 kPa, a temperature of the heat medium of 130° C., and an internal temperature of 100° C. With confirming the concentration of furancarboxylic acid in the pot liquid by means of GC, when it reached 6,800 ppm by mass, the distillation was terminated. As a result, a solid matter was not found in the flask. The amount of the pot residue was 7.5 g.

### Comparative Example 15

The same procedures as in Example 21 were carried out, except that when the concentration of furancarboxylic acid in the pot liquid reached 12,000 ppm by mass, the distillation was terminated. The amount of the pot residue after the distillation was 4.0 g, and 56 mg of a solid matter was formed in the flask after termination of the distillation.

### Example 22

### Production of Furan by Decarbonylation Reaction of Furfural

In a glass-made reaction tube having an inside diameter of 6 mm, 0.75 g of a supported Pd catalyst (1% by mass Pd-1% by mass K/ZrO₂) which had been crushed to a size of 0.6 mm or less was filled, and the temperature of the catalyst was increased to 231° C. under circulation of 2.25 mmol/h of hydrogen and 85.71 mmol/h of nitrogen. A furfural composition purified by the same method as in Example 1 was passed through a vaporizer heated at 182° C. and vaporized, followed by feeding at a flow rate of 36.22 mmol/h, to commence a decarbonylation reaction. At that time, a ratio of hydrogen to the furfural compound was 0.062. A reaction pressure was 0.1 MPa in terms of an absolute pressure.

A part of the reaction gas obtained from an outlet of the reaction tube was introduced into a gas chromatograph, thereby quantitating the furan compound, carbon monoxide, nitrogen, and other products.

For the gas chromatographic analysis of inorganic gases, such as carbon monoxide, nitrogen, etc., a thermal conductivity detector was used as a detector, and a packed column filled with Molecular Sieve 13X (mesh 60/80) and having a column length of 3 m was used as a column. The analysis was carried out by setting a temperature of each of the sample introducing part and the detection part to be 90° C., a temperature of the column to be 70° C., and a current value to be flown into the detection part to be 70 mA.

For the gas chromatographic analysis of organic gases, such as furfural, furan, etc., a thermal conductivity detector was used as a detector, and a packed column filled with Termon-1000 (medium polarity) and having a column length of 3 m was used as a column. The analysis was carried out in such a manner that a temperature of the sample introducing part was set to be 200° C.; a temperature of the detection part was set to be 220° C.; a temperature of the column was increased at a rate of 3° C./min from 80° C. to 110° C., after reaching 110° C., the temperature was increased at a rate of 5° C./min to 225° C., and after reaching 225° C., the temperature was kept for 17 minutes; and a current value to be flown into the detection part was set to be 80 mA.

A furfural conversion rate (%) and a furan selectivity (%) were determined.

Furfural conversion rate (%)=[1−{(Residual amount of furfural compound after reaction (mol))/(Feed amount of furfural compound (mol))}]×100

Furan selectivity (%)=[{(Yield of furan compound (%))/(Conversion of furfural compound (%))}×100=[{(Formation amount of furan compound (mol))/(Feed amount of furfural compound (mol))}×100/(Furfural conversion rate (%))]×100

As a result of performing the decarbonylation reaction under the above-described condition, 12 hours after commencing the reaction, the furfural conversion rate was 93.3%, and the furan selectivity was 98.8%.

It is noted from Examples 1 and 2 and Comparative Example 1 that when the furfural dimer and the furancarboxylic acid are excessively concentrated, the formation of a solid matter is promoted.

It is noted from Examples 3 to 5 that when a high-temperature heat medium is used, the high-boiling component increases, and the concentration of furancarboxylic acid increases due to contamination of oxygen; however, by appropriately controlling the furfural dimer and the furancarboxylic acid, the formation of a solid matter can be inhibited. That is, it is noted that when the concentration of furancarboxylic acid is low, and the acid value is low, even in the same concentration of furfural dimer, it is possible to inhibit the formation of a solid matter.

In addition, it is noted from Tables 3 and 4 that when the heating is performed in a state where the concentration of furfural dimer exceeds 5,000 ppm by mass, and the concentration of furancarboxylic acid exceeds 8,000 ppm by mass, the formation of a solid matter is remarkably promoted. Furthermore, it is noted from Table 5 that when a small amount of the furfural dimer or furancarboxylic acid is present in the composition, a side reaction to be caused due to an elution component from the material can be inhibited.

It is confirmed from Tables 3 to 5 that by heating the composition containing furfural, a solid matter is formed, or a side reaction is generated. These Examples and Comparative Examples are those supposing the conditions of a column bottom of a distillation column in the production method of furfural according to the present invention. Accordingly, even in the case of distilling the composition containing furfural in a distillation column under the same conditions as in these Examples and Comparative Examples, to produce furfural, it may be assumed that equal effects to the effects confirmed through comparison between these Examples and Comparative Examples are revealed as the tendency.

While the invention has been described in detail and with reference to specific embodiments thereof, it will be apparent to one skilled in the art that various changes and modifications can be made therein without departing from the spirit and scope thereof. It is to be noted that the present application is based on a Japanese patent application filed on May 8, 2014 (Japanese Patent Application No. 2014-096968), and the contents are incorporated herein by reference.

1. A method for producing furfural comprising distilling a composition comprising furfural by a distillation column to obtain furfural, wherein a concentration of a furfural dimer in a column bottom liquid of the distillation column is 20 ppm by mass to 5,000 ppm by mass.

2. The method for producing furfural according to claim 1, wherein a concentration of furancarboxylic acid in the column bottom liquid of the distillation column is 50 ppm by mass to 8,000 ppm by mass.

3. The method for producing furfural according to claim 1, including a step of prior to distilling the composition comprising furfural by the distillation column, concentrating a compound having a higher boiling point than furfural in crude furfural obtained after bringing the crude furfural into contact with an anion exchange resin and/or a basic compound in advance, to obtain the composition comprising furfural.

4. The method for producing furfural according to claim 1, wherein a concentration of furfural in the composition comprising furfural is 87.0% by mass or more and 99.0% by mass or less.

5. The method for producing furfural according to claim 1, wherein a temperature of the column bottom liquid of the distillation column for distilling the composition comprising furfural is 60 to 180° C.

6. The method for producing furfural according to claim 1, wherein an acid value of the column bottom liquid of the distillation column for distilling the composition comprising furfural is 10 mg-KOH/g or less.

7. A method for producing furan comprising feeding the furfural obtained by the method for producing furfural according to claim 1 into a reactor; performing a decarbonylation reaction in the presence of a catalyst to form furan; and extracting a mixed gas containing the furan as a main component from an outlet of the reactor.
